SEQUENCE LISTING (4 sequences)

SEQ ID NO: 1 - DNA, 20 bp, Artificial Sequence
  TNF_F
acaaggcaatttcacttcca

SEQ ID NO: 2 - DNA, 20 bp, Artificial Sequence
  Il1b_R
agctgatgtgcaaagacacc

SEQ ID NO: 3 - DNA, 20 bp, Artificial Sequence
  Il1b_F
ttgtgggagacagacggtgc

SEQ ID NO: 4 - DNA, 23 bp, Artificial Sequence
  TNF_R
gattggggtttgatgtgcttcat

(19) United States
(12) Patent Application Publication
(10) Pub. No.: US 2024/0216317 A1
(43) Pub. Date: Jul. 4, 2024

(54) COMPOSITION FOR PREVENTING, AMELIORATING OR TREATING INFLAMMATORY BOWEL DISEASE COMPRISING COMPOUND ISOLATED FROM ISODON EXCISUS AS EFFECTIVE COMPONENT

(71) Applicant: HEALINNOLS INC., Daejeon (KR)

(72) Inventors: Jin Hee KIM, Daejeon (KR); Minju KANG, Daejeon (KR); Eunji LEE, Daejeon (KR)

(21) Appl. No.: 17/916,240

(22) PCT Filed: Jun. 14, 2022
(86) PCT No.: PCT/KR2022/008376

§ 371 Date: Sep. 30, 2022

(30) Foreign Application Priority Data
Feb. 21, 2022 (KR) 10-2022-0022078

(51) Int. Cl. A61K 31/216; A23K 20/111; A61K 36/53; A61P 1/00

(52) U.S. Cl. CPC A61K 31/216; A23K 20/111; A61K 36/53; A61P 1/00; A61K 2236/33; A61K 2236/39; A61K 2236/51

(57) ABSTRACT

A method for treating inflammatory bowel disease includes administering to a subject in need thereof a composition comprising a compound represented by Chemical Formula 1 or a pharmaceutically or sitologically acceptable salt thereof. The compound represented by Chemical formula 1 not only can suppress the inflammatory responses by inhibiting the phosphorylation activity of RIPK2 and exhibits an effect of regenerating intestinal mucus layer in accordance with recovery of the tight junction-related proteins as an intercellular junction factor but also has an effect of reducing the transcription amount of proinflammatory cytokine mRNA in zebra fish animal model of inflammatory bowel disease. Thus, it can be advantageously used for a pharmaceutical product, a functional health food product, or an animal feed for preventing, ameliorating, or treating inflammatory bowel disease.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/008376 filed on Jun. 14, 2022, which claims priority to the benefit of Korean Patent Application No. 10-2022-0022078 filed in the Korean Intellectual Property Office on Feb. 21, 2022, the entire contents of which are incorporated herein by reference.

### BACKGROUND

### 1. Technical Field

The present invention relates to a composition for preventing, ameliorating or treating inflammatory bowel disease comprising a compound isolated from Isodon excisus as an effective component.

### 2. Background Art

Inflammatory bowel disease (IBD) is one type of autoimmune diseases and it is characterized by repeated remission and relapse of abnormal chronic inflammation in intestinal tract. Inflammatory bowel disease is generally divided into two types, i.e., ulcerative colitis (UC) and Crohn's disease (CD). In ulcerative colitis, the inflammation condition is limited to the large intestine, while Crohn's disease is characterized in that the inflammation occurs in the entire digestive tract, i.e., from mouth to anus. Although the inflammatory bowel disease is a disorder that is found at high rate mainly among westerners, the disease incidence frequency in South Korea is now dramatically higher than before, i.e., it has increased by 2 times or more during last 10 years, due to the shift to western pattern diet. Most people with inflammatory bowel disease have been diagnosed in their 30s and 40s. However, in recent years, people in their 20s are also found to develop inflammatory bowel disease. Furthermore, it is known that patients suffering from inflammatory bowel disease have a higher risk of having colon cancer, i.e., about 2 to 11 times higher than healthy people.

The exact cause of inflammatory bowel disease remains unclear, but, once gut microbes enter the intestines through intestinal mucus layer damaged by genetic or environmental cause, or abnormal immune factors, intestinal immune cells are activated. Since overactivation of the immune cells can cause an abnormality in immune cell regulatory system to yield a development of chronic inflammation, inflammatory bowel disease may consequently occur.

At the present moment, there are no reports describing the pharmaceuticals that can completely cure inflammatory bowel disease. However, those generally prescribed for treating inflammatory bowel disease include 5-aminosalicylic acid (i.e., anti-inflammation agent, and categorized into sulfasalazine, mesalazine, and the like depending on the type of formulation), adrenocortical steroids, immunomodulating agents (thiopurine), and anti-TNA-α biologic agents, which are prescribed and administered in order depending on severity of the symptoms. Meanwhile, those pharmaceuticals currently prescribed for inflammatory bowel disease exhibit various side effects. Main object of treating inflammatory bowel disease is to reduce inflammatory responses and promote the healing and regeneration of damaged tissues. As such, it is currently desired to develop a new therapeutic agent having both the anti-inflammation activity and cell-regeneration effect.

Incidence of inflammatory bowel disease is accompanied by inflammation responses that are related with a pro-inflammatory cytokines, such as TNFα, IL-6, and IL-8. In particular, TNFα is found in large amount in intestinal epithelial cells of a patient with ulcerative colitis, and infliximab, which is an anti-TNFα antibody, is currently prescribed and used for treating not only ulcerative colitis but also Crohn's disease. However, such antibody therapeutic agent has a problem like lack of response or resistance to pharmaceuticals which is caused by mutation in a region to which the antibody binds, and the agent is highly costly. TNF-α is induced by NF-κB, which is a transcription factor, and the activation pathway of NF-κB is the most important signal transduction pathway in innate immunity.

Meanwhile, NOD1 and NOD2 as NOD (nucleotide-binding oligomerization domain) protein are a sensor for recognizing microbial materials originating from bacterial peptidoglycan, and they play a key role in innate immune responses. In particular, mutation in NOD2 is widely known as a biomarker related to the sensitivity for inflammatory bowel disease. One of the main results caused by signal transduction involving NOD protein is the activation of NF-κB. Abnormal NOD-related signal transduction induces overactivation of NF-κB. The signal transduction pathway is mediated by RIPK2 (receptor-interacting serine/threonine-protein phosphorylase 2), which is a phosphorylating enzyme. As it has been reported that overactive RIPK2 is found in a patient with Crohn's disease as inflammatory bowel disease, it is recognized that RIPK2 is not only a potential target for the treatment of inflammatory bowel disease but also a core target protein for studying the pathogenesis of inflammatory bowel disease.

Although many studies are being carried out by having RIPK2 as a therapeutic target of various diseases, there are still few studies which take RIPK2 as a target for developing a therapeutic agent for inflammatory bowel disease.

Meanwhile, in Korean Patent Registration No. 1661423, an anti-oxidation and anti-inflammation composition comprising compound isolated from Isodon excisus and a method of extracting the compound are disclosed, and, in Korean Patent Registration No. 2117487, a pharmaceutical composition for preventing or treating inflammatory bowel disease comprising broussochalcone A as an effective component is disclosed. However, the composition of the present invention, which is used for preventing, ameliorating or treating inflammatory bowel disease by comprising a compound isolated from Isodon excisus as an effective component, has not been described.

### SUMMARY

The present invention is devised under the circumstances that are described in the above, and the present invention provides a composition for preventing, ameliorating or treating inflammatory bowel disease comprising a compound isolated from Isodon excisus as an effective component. According to the finding that the compound represented by Chemical formula 1 of the present invention not only exhibits an inhibitory activity on phosphorylation of RIPK2 and enhances the expression amount of tight junction protein (Zonula occludens-1; ZO-1, occludin and claudin1), which is an intercellular junction protein in intestinal epithelial cell, but also reduces the transcription amount of proinflammatory cytokine mRNA in zebra fish animal model of inflammatory bowel disease, the present invention is completed.

To achieve the object described in the above, the present invention provides a pharmaceutical composition for preventing or treating inflammatory bowel disease comprising a compound represented by Chemical formula 1 or a pharmaceutically acceptable salt thereof as an effective component.

The present invention further provides a functional health food composition for preventing or ameliorating inflammatory bowel disease comprising a compound represented by Chemical formula 1 or a salt thereof acceptable for use in foods as an effective component.

The present invention still further provides an animal feed composition for preventing or ameliorating inflammatory bowel disease comprising a compound represented by Chemical formula 1 or a pharmaceutically acceptable salt thereof as an effective component.

The present invention relates to a composition for preventing, ameliorating or treating inflammatory bowel disease comprising a compound isolated from Isodon excisus as an effective component. The compound represented by Chemical formula 1 of the present invention not only can suppress inflammatory responses by inhibiting the phosphorylation activity of RIPK2 and exhibits an effect of regenerating intestinal mucus layer in accordance with recovery of the tight junction-related proteins as an intercellular junction factor but also has an effect of reducing the transcription amount of proinflammatory cytokine mRNA in zebra fish animal model of inflammatory bowel disease.

### BRIEF DESCRIPTION OF THE DRAWINGS

FIG. 1 shows the result of Western blot for determining the effect of inhibiting phosphorylation activity of RIPK2 in which the inhibition is caused by a treatment with the compound represented by Chemical formula 1 of the present invention.

FIG. 2 shows the result of immunofluorescence microscopy analysis for determining a change in expression amount of ZO-1, i.e., a tight junction-related protein, in intestinal epithelial wall model, in which the change in expression amount was determined to examine the effect of healing intestinal lining by the compound represented by Chemical formula 1 of the present invention. ** and *** indicate that, compared to the expression amount of ZO-1 in the model group of ulcerative colitis caused by DSS treatment, the expression amount of ZO-1 is statistically significantly higher in the positive control (5-ASA) and also in the group treated with the compound represented by Chemical formula 1 of the present invention, in which ** means p<0.01 and *** means p<0.001. ns means that there is no statistically significant difference.

FIG. 3 shows the result of immunofluorescence microscopy analysis for determining a change in expression amount of occludin, i.e., a tight junction-related protein, in intestinal epithelial wall model, in which the change in expression amount was determined to examine the effect of healing intestinal lining by the compound represented by Chemical formula 1 of the present invention. * and ** indicate that, compared to the expression amount of occludin in the model group of ulcerative colitis caused by DSS treatment, the expression amount of occludin is statistically significantly higher in the positive control (5-ASA) and also in the group treated with the compound represented by Chemical formula 1 of the present invention, in which * means p<0.05 and ** means p<0.01.

FIG. 4 shows the result of immunofluorescence microscopy analysis for determining a change in expression amount of claudin1, i.e., a tight junction-related protein, in intestinal epithelial wall model, in which the change in expression amount was determined to examine the effect of healing intestinal lining by the compound represented by Chemical formula 1 of the present invention. ** indicates that, compared to the expression amount of claudin1 in the model group of ulcerative colitis caused by DSS (Dextran Sulfate Sodium) treatment, the expression amount of claudin1 is statistically significantly higher in the positive control (5-ASA) and also in the group treated with the compound represented by Chemical formula 1 of the present invention, in which p<0.01.

FIG. 5 shows the result of determining the effect of inhibiting the expression of (B) TNF-α and (C) IL-1β, which are proinflammatory cytokines, in (A) zebra fish animal model of inflammatory bowel disease, by the compound represented by Chemical formula 1 of the present invention. * and ** indicate that, compared to the expression amount of TNF-α or IL-1β in the model group of ulcerative colitis caused by DSS treatment, the expression amount of TNF-α and IL-1β is statistically significantly lower in the positive control (5-ASA) and also in the group treated with the compound represented by Chemical formula 1 of the present invention, in which * means p<0.05 and ** means p<0.01. ns means that there is no statistically significant difference.

FIG. 6 shows the result of determining the relative change in body weight in each treatment group, in which the body weight is examined for animal model of DSS-induced inflammatory bowel disease.

FIG. 7 shows the result of determining a change in disease activity index (DAI) in animal model of DSS-induced inflammatory bowel disease, in which the change is determined in accordance with the administration of the compound represented by Chemical formula 1 of the present invention.

FIG. 8 shows (A) photographic image and (B) graph representing a change in the colon length of animal model of DSS-induced inflammatory bowel disease, in which the change is followed in accordance with the administration of the compound represented by Chemical formula 1 of the present invention.

### DETAILED DESCRIPTION

The present invention relates to a pharmaceutical composition for preventing or treating inflammatory bowel disease comprising a compound represented by the following Chemical formula 1 (i.e., 3-[3,4-dihydroxyphenyl]acrylic acid 1-[3,4-dihydroxyphenyl]-2-methoxycarbonylethyl ester) or a pharmaceutically acceptable salt thereof as an effective component.

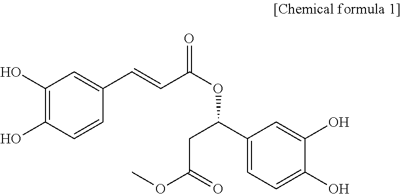 [SMILES: COC(=O)C[C@H](OC(=O)/C=C/c1ccc(O)c(O)c1)c1ccc(O)c(O)c1]

The inflammatory bowel disease is preferably any one selected from the group consisting of ulcerative colitis, Crohn's disease, ischemic colitis, intestinal Behcet's disease, bleeding rectal ulcer, and pouchitis, but it is not limited thereto.

As described herein, the expression, “pharmaceutically acceptable salt” means any kind of organic or inorganic addition salt of the compound of Chemical formula, which is relatively nontoxic to a patient and does not exhibit any side effects derived from the salts to compromise the beneficial effects of the compound. As a free acid, inorganic acid, organic acid, nontoxic salt, or the like can be used, for example. Examples of the inorganic acid that can be preferably used include hydrochloric acid, phosphoric acid, sulfuric acid, nitric acid, and tartaric acid. Examples of the organic acid that can be preferably used include methane sulfonic acid, p-toluene sulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid.

Acid addition salt of the aforementioned inorganic acid or organic acid can be prepared by a common method, for example, by dissolving a compound in an excessive amount of aqueous acid solution and precipitating the salt in water-miscible organic solvent like methanol, ethanol, acetone, and acetonitrile. The compound may be heated with acid or alcohol in water, all in same molar amount, and the mixture may be subsequently dried by evaporation or the precipitated salt may be subjected to suction filtration.

Examples of the nontoxic salt that can be preferably used include sulfate, pyrosulfate, bisulfate, sulfite, bisulfite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonates, toluenesulfonate, chlorobenzenesulfonate, xylenesulfonate, phenylacetate, phenylpropionate, phenylbutyrate, citrate, lactate, (3-hydroxybutyrate, glycolate, malate, tartrate, methanesulfonate, propanesulfonate, naphthalene-1-sulfonate, naphthalene-2-sulfonate, and mandelate.

As described herein, the term “preventing” means all kinds of activities of inhibiting or delaying the onset of inflammatory bowel disease by administration of the pharmaceutical composition of the present invention, and the term “treat” means all kinds of activities of ameliorating or positively changing the symptoms of an individual, who is either suspected or known to have inflammatory bowel disease, by administration of the pharmaceutical composition of the present invention.

Route of administration for effective administration of the pharmaceutical composition of the present invention is not particularly limited, and a suitable route of administration can be selected and applied for the patient. For example, oral administration, rectal administration, transdermal administration, parenteral administration (i.e., subcutaneous, muscular, and blood vessel administration), dural administration, topical administration, administration by inhalation, and other administration methods may be used.

With regard to the dosage form of the pharmaceutical composition of the present invention, administration can be made by using various types of dosage forms according to a common method that is well known in the field of pharmaceutical preparation. Examples of the dosage form that can be suitably used include an oral formulation like powder, granule, tablet, troche, dispersion, suspension, liquid, capsule, emulsion, syrup, and aerosol, formulation for external use, suppository, an injection solution, a patch, and other suitable formulations.

The pharmaceutical composition of the present invention may further comprise a suitable carrier, vehicle, or diluent which is typically used for preparing a pharmaceutical composition. Examples of the suitable carrier, vehicle, or diluent that may be comprised in the pharmaceutical composition include various compounds or mixtures like lactose, dextrose, sucrose, sorbitol, mannitol, xylitol, erythritol, maltitol, starch, acacia gum, alginate, gelatin, calcium phosphate, calcium silicate, cellulose, methyl cellulose, non-crystalline cellulose, polyvinyl pyrrolidone, water, methylhydroxybenzoate, propylhydroxybenzoate, talc, magnesium stearate, and mineral oil.

For producing a formulation, the preparation can be made by using a diluent or a vehicle such as filler, bulking agent, binding agent, moisturizing agent, disintegrating agent, or surfactant that are commonly used for producing a formulation. Examples of the solid formulation for oral administration include a tablet, a pill, a powder, a granule, and a capsule. The solid formulation is produced by mixing the microbe or microbial vesicle with one or more vehicles such as starch, calcium carbonate, sucrose, lactose, or gelatin. Furthermore, in addition to those simple vehicles, a lubricating agent such as magnesium stearate or talc is also used. As for the liquid formulation for oral administration, a suspension, a solution formulation for internal use, an emulsion, a syrup formulation, or the like can be mentioned. Other than water or liquid paraffin commonly used as a simple diluent, various kinds of a vehicle such as moisturizing agent, sweetening agent, aromatic agent, or preservatives may be included. In a formulation for parenteral administration, a sterilized aqueous solution, a non-aqueous solvent, a suspending agent, an emulsifying agent, a freeze-dried formulation, and a suppository formulation may be included. As a non-aqueous solvent or a suspending agent, propylene glycol, polyethylene glycol, or vegetable oil such as olive oil, and injectable ester such as ethylolate can be used. As a base for a suppository formulation, witepsol, macrogol, tween 61, cacao fat, laurin fat, glycerol, gelatin, or the like can be used.

The pharmaceutical composition of the present invention may be administered in a pharmaceutically effective amount. As described herein, the expression “pharmaceutically effective amount” means an amount sufficient for preventing or treating a disorder at reasonable benefit-risk ratio that can be applied for clinical prophylaxis or treatment. The effective dose level may be determined based on a type or severity of a disorder, activity of a pharmaceutical, age, body weight, health state, sex, and sensitivity to a pharmaceutical of a patient, administration period, route of administration, and excretion ratio of the composition of the present invention, time period for therapy, elements including a pharmaceutical used in combination of the composition of the present invention, and other elements that are well known in the medical field. The pharmaceutical composition of the present invention can be administered as a separate therapeutic agent, or it can be used in combination with other therapeutic agent. It can be administered in order or simultaneously with a conventional therapeutic agent. It can be also administered as single-dose or multi-dose. It is important to administer an amount which allows obtainment of the maximum effect with minimum dose while considering all of the aforementioned elements without having any side effect.

With regard to the administration frequency of the composition of the present invention, the administration can be made once a day, or several times a day, i.e., divided portions of daily dose, although it is not particularly limited thereto. The dose may vary depending on age, body weight, sex, form of administration, health state, and severity of disorder. For an adult patient with body weight of 70 kg as an example, the dose may be in the range of preferably 0.1 to 300 mg/day, more preferably 0.5 to 100 mg/day, and even more preferably 1 to 60 mg/day, but the scope of the present invention is not limited by them in any aspect.

The present invention further relates to a functional health food composition for preventing or ameliorating inflammatory bowel disease comprising a compound represented by the following Chemical formula 1 or a salt thereof acceptable for use in foods as an effective component.

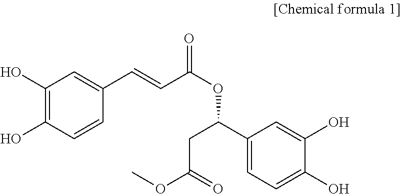 [SMILES: COC(=O)C[C@H](OC(=O)/C=C/c1ccc(O)c(O)c1)c1ccc(O)c(O)c1]

The effective component is preferably prepared in any formulation selected from powder, granule, pill, tablet, capsule, candy, syrup, and beverage, but it is not limited thereto.

When the compound represented by Chemical formula 1 or a pharmaceutically acceptable salt thereof is used in the functional health food composition, it may be either directly added or used with other food products or food compounds in the functional health food composition, and it may be suitably used according to a common method. Mixing amount of the effective compound can be suitably determined depending on the purpose of use.

The functional health food composition of the present invention may further comprise a component which is generally used for preparing a food product and acceptable for use in foods. When the composition is prepared as a beverage, for example, one or more components of citric acid, high fructose corn syrup, sucrose, glucose, acetic acid, malic acid, and fruit juice may be further comprised in addition to the compound represented by Chemical formula 1 of the present invention or a pharmaceutically acceptable salt thereof.

The amount to be comprised as an effective component of the functional health food composition of the present invention can be suitably selected based on the age, sex, body weight, health state, symptoms of disorder, or the like of a person who desires to consume the functional health food composition for preventing or ameliorating inflammatory bowel disease. Preferably, for an adult, it is comprised in an amount of 0.01 to 10.0 g or so per day. By consuming a food with the effective component contained in such amount, the effect of preventing or ameliorating inflammatory bowel disease can be obtained.

The present invention still further relates to an animal feed composition for preventing or ameliorating inflammatory bowel disease comprising a compound represented by the following Chemical formula 1 or a pharmaceutically acceptable salt thereof as an effective component.

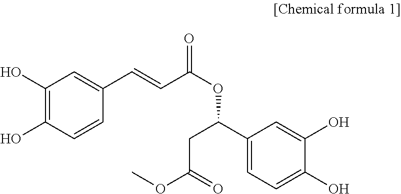 [SMILES: COC(=O)C[C@H](OC(=O)/C=C/c1ccc(O)c(O)c1)c1ccc(O)c(O)c1]

The compound represented by Chemical formula 1 and a pharmaceutically acceptable salt thereof are the same as those described in the above, and they can be added to an animal feed composition under the purpose of preventing or ameliorating inflammatory bowel disease. The animal feed composition may also comprise an animal feed additive. The animal feed additive described in the present invention corresponds to a complementary animal feed described under Control of Livestock and Fish Feed Act.

As described herein, the term “animal feed” may mean any natural or artificial meal, single meal, or the like which is eaten, consumed, and digested by an animal or appropriate for such act, or components of such single meal. Type of the animal feed is not particularly limited, and any animal feed commonly used in the corresponding technical field can be used. Non-limiting examples of the animal feed include plant feed like grains, roots and fruits, by-products obtained after food processing, algae, fibers, by-products of pharmaceutical preparation, oils and fats, starches, pulps, and grain by-products; and animal feed like proteins, inorganic materials, oils and fats, mineral oils, oils and fats, single cell proteins, zooplanktons, and food stuffs. They may be used either singly or as a mixture of two or more kinds thereof.

Hereinbelow, the present invention is explained in greater detail in view of the Examples. However, the following Examples are given only for specific explanation of the present invention and it would be evident to a person who has common knowledge in the pertinent art that the scope of the present invention is not limited by them.

### EXAMPLES

### Example 1. Isolation of Compound Represented by Chemical Formula 1 from Isodon excisus (Max.) Kudo

Isodon excisus (Max.) Kudo (2.5 kg), which has been dried after being collected from mountain Jiri in South Korea, was extracted with methanol for 48 hours followed by concentration under reduced pressure and further extraction with ethyl acetate. The ethyl acetate layer was then concentrated and dissolved in a small amount of mixture solvent, in which chloroform and methanol are admixed with each other (100:1). After carrying out silica gel column chromatography (20-230 mes, Merk), active fractions were concentrated. The concentrated active fractions were dissolved in methanol and subjected to Sephadex LH-20 gel column chromatography to collect the active fractions.

The resulting active fractions concentrated above were then subjected to high pressure liquid chromatography (column: C18, flor rate: 1.5 ml/minute, detection at 220 nm) which uses water and acetonitrile as a solvent for elution (water:acetonitrile—80:20 to 100:0 over 30 minutes) to separate the active factions. The solvent was removed by drying under reduced pressure, and then the resulting residues (i.e., compound represented by Chemical formula 1; 3-[3,4-dihydroxyphenyl]acrylic acid 1-[3,4-dihydroxyphenyl]-2-methoxycarbonylethyl ester) were subjected to freeze-drying. After that, ¹H, ¹³C and HMBC NMR analyses were carried out to identify the structure of the compound represented by Chemical formula 1 (Journal of Natural Products, 2001, vol. 64, No. 5, pp. 659 to 660).

### Example 2. Determination of Inhibitory Activity on RIPK2 Phosphorylation Using Western Blot

Caco-2 cells were adjusted to have density of 2×10⁵ cells/ml. Suspension of the cells (0.5 ml) was aliquoted in apical side membrane phase of a transwell insert 12-well plate. To the basolateral side phase, 1.5 ml of culture medium were added. While replacing the medium with fresh one, once per 2 or 3 days, culture was continued for 21 days approximately, i.e., till to have cell division and suitable TEER (transepithelial electrical resistance) value of monolayer.

For measuring the electric resistance (TEER value, Ω·cm²) between the apical side and basolateral side, EVOM resistance meter (EVOM2, WPI) was employed.

Once the TEER value of Caco-2 cells is 200 Ω·cm² or higher, MDP inside the insert was treated for 24 hours with the compound represented by Chemical formula 1 of the present invention. After washing Caco-2 cells inside the insert with 1×PBS, they were treated with trypsin. After centrifuge and washing with cold 1×PBS, Caco-2 cells were dissolved by adding buffer solution for degradation (50 mM Tris-HCl (pH 8.0), 5 mM EDTA, 15 mM NaCl, 1% NP-40, 1 mM PMSF) followed by centrifuge for 20 minutes at 12,000 rpm, 4° C. Only the supernatant was then collected and protein quantification was carried out by using BCA protein assay kit. After adding a sample buffer solution (1 M Tris-HCl (pH 6.8), 50% glycerol, 10% SDS, 1% bromophenol blue, and 5% mercaptoethanol) followed by mixing, the mixture was boiled for 5 minutes at 100° C. Then, electrophoresis on 15% acrylamide gel was carried out and transfer to an NC membrane was carried out for 1 hour at 100 V. The membrane was separated, blocked for 1 hour with skim milk, and added with primary antibody which has been diluted in 5% BSA. After overnight culture, washing was carried out 3 times with 1× TBS-T, for 10 minutes for each, and HRP-conjugated secondary antibody, which has been diluted in 5% skim milk, was added thereto to have a reaction for 2 hours. Washing was carried out 3 times with 1×TBS-T, for 10 minutes for each, and then a change in the amount of protein expression was examined by using ECL (enhanced chemiluminescence) solution.

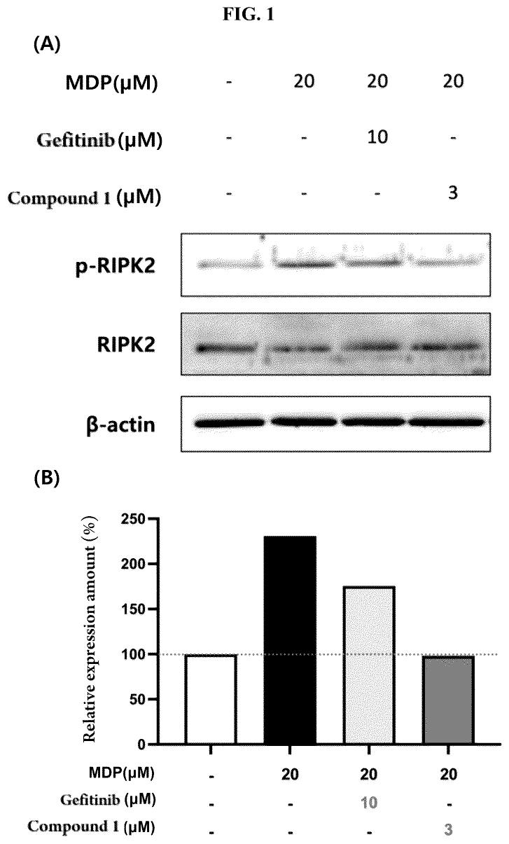

As a result, it was found that, even at lower concentration, the compound represented by Chemical formula 1 of the present invention can inhibit the activation of RIPK2. Based on this experimental result, it was recognized that the compound represented by Chemical formula 1 of the present invention has an inhibitory activity on RIPK2 phosphorylation, which is at least 2 times higher than gefitinib (positive control) known as an RIPK2 inhibitor in real cells (FIG. 1).

### Example 3. Evaluation of Effect of Healing Intestinal Lining Along with Recovery of Tight Junction-Related Proteins

### (1) Establishment of Intestinal Epithelial Wall Model

Caco-2 cell line (human intestinal epithelial cell line) and RAW 264.7 cell line, which is macrophage cells derived from mouse, were cultured at 37° C., 5% CO2 conditions by using DMEM (Dulbecco's Modified Eagle Medium, #11995065, Gibco) containing 10% FBS (fetal bovine serum, #16000044, Gibco) and 1% streptomycin/penicillin (#15140122, Gibco). For culturing the cells, the medium exchange was carried out every 2 to 3 days, and, once the cells are grown to the level of 80% in cell culture dish (#353003, Falcon), they were maintained by subculture.

The Caco-2 cells were adjusted to have density of 2×10⁵ cells/ml. Suspension of the cells (0.5 ml) was aliquoted in apical side membrane phase of a transwell insert 12-well plate. To the basolateral side phase, 1.5 ml of culture medium were added. While replacing the medium with fresh one, once per 2 or 3 days, culture was continued for 21 days approximately, i.e., till to have cell division and suitable TEER (transepithelial electrical resistance) value of monolayer. For measuring the electric resistance (TEER value, Ω·cm²) between the apical side and basolateral side, EVOM resistance meter (EVOM2, WPI) was employed.

Once the TEER value of Caco-2 cells is 200 Ω·cm² or higher, the RAW 264.7 cells were adjusted to have density of 5×10⁵ cells/ml. Suspension of the RAW 264.7 cells (2 ml) was aliquoted in 12-well plate followed by culture for 24 hours. The medium for culturing Caco-2 cells was replaced with fresh one, and, by adding the Caco-2 cells which have been cultured in the above to the 12-well plate containing RAW 264.7 cells, the intestinal epithelial wall model was obtained.

### (2) Immunofluorescence Microscopy Analysis of Tight Junction-Related Proteins

Caco-2 cells were adjusted to have density of 2×10⁵ cells/ml. Suspension of the cells (0.5 ml) was aliquoted in apical side membrane phase of a transwell insert 12-well plate. To the basolateral side phase, 1.5 ml of culture medium were added. While replacing the medium with fresh one, once per 2 or 3 days, culture was continued for 21 days approximately, i.e., till to have cell division and suitable TEER (transepithelial electrical resistance) value of monolayer. For measuring the electric resistance (TEER value, Ω·cm²) between the apical side and basolateral side, EVOM resistance meter (EVOM2, WPI) was employed.

Culture medium of the Caco-2 cells, which have been cultured for 21 days inside the insert, was removed, and it was replaced with culture medium containing 3% DSS; 3% DSS+compound represented by Chemical formula 1 of the present invention (0.3, 1, or 3 μM); or 3% DSS+5-ASA (500 μM) followed by culture for 24 hours.

After that, the Caco-2 cells inside the insert were washed twice with 1×PBS and fixed for 10 minutes at room temperature by treating them with 4% paraformaldehyde. After washing them again 3 times with 1×PBS, cell membrane permeation was carried out for 10 minutes at room temperature by treatment with 0.1% Triton-X 100. The cells were then washed 3 times with 1×PBS.

Then, blocking was carried out at room temperature by treatment with 1% BSA for 30 minutes. After treating them with primary antibody followed by overnight treatment for adhesion, the cells were washed 3 times with 1×PBS, treated with fluorescent-labeled secondary antibody, and then allowed to have adhesion for 1 hour at room temperature while blocking the light.

Thereafter, the cells were washed 3 times with 1×PBS for 5 minutes and treated for 5 minutes with Hoechst 33342 for staining the nucleus. After washing 3 times with 1×PBS, the membrane was separated using a scalpel and then applied on a glass slide. After the mounting with ProLong™ Gold Antifade reagent, it was examined by confocal fluorescence microscopy.

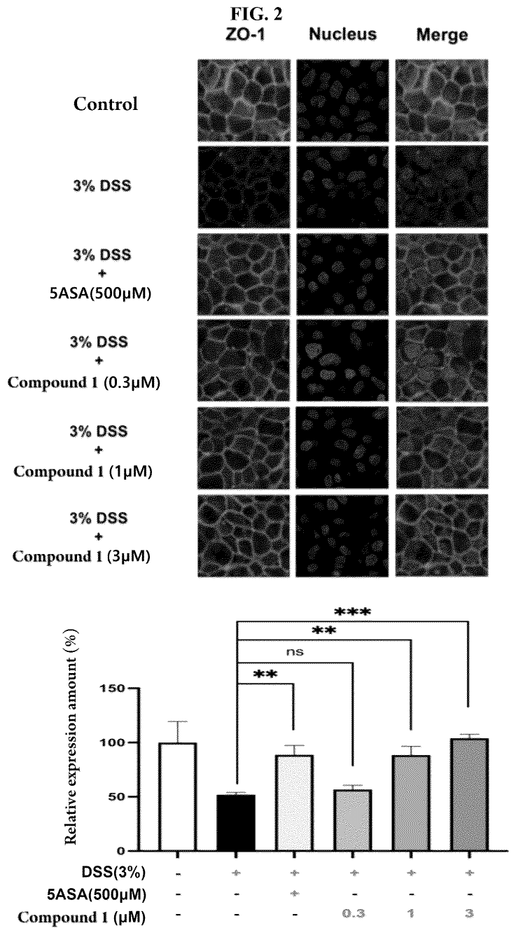

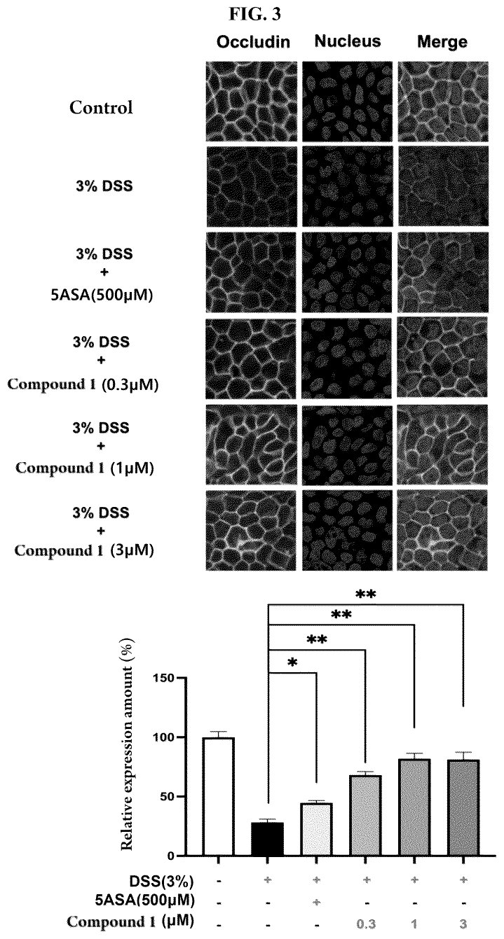

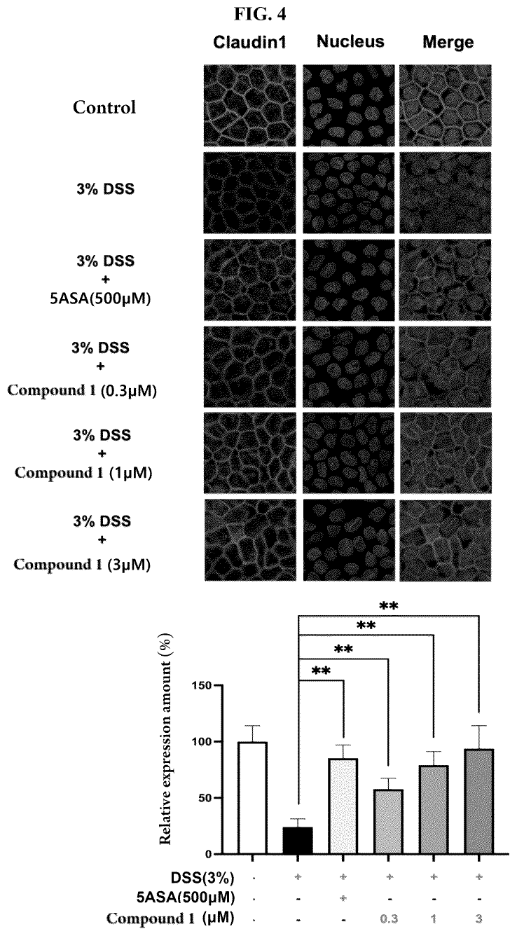

As a result of the immunofluorescence microscopy analysis for determining a change in the expression amount of tight junction-related proteins in Caco-2 cells using intestinal epithelial wall model, it was found that ZO-1, occludin, and claudin1, all tight junction-related protein, are recovered in the inflammatory bowel disease (i.e., ulcerative colitis) induced by 3% DSS (dextran sulfate sodium). It was also found that the effect of regenerating mucus layer by the subject material of the present invention, which is shown in accordance with the recovery of tight junction-related proteins as an intercellular junction protein, is better than the effect of 5-ASA, which is currently used as a therapeutic agent of inflammatory bowel disease (FIGS. 2 to 4).

### Example 4. Establishment of Zebra Fish Animal Model of Inflammatory Bowel Disease and Analysis of mRNA Expression Amount of Proinflammatory Cytokines

As a zebra fish animal model of inflammatory bowel disease, wild-type zebra fish (Danio rerio: AB) was used, which was kept in an exclusive breeding facility at conditions including light/dark cycle of 14 hours/10 hours and temperature of 28° C. All the animal experiments were carried out according to the protocol of Ethics Committee for Animal Expectation, Healinno-LS (Registration No.: AEC-20210720-0001), and all the materials were sterilized first before they are used for experiment.

According to a treatment with 0.5% DSS (dextran sulfate sodium, M.W. 36,000-50,000, #160110, MP), which induces “ulcerative colitis” as inflammatory bowel disease by creating a damage in colonic epithelial cells, a zebra fish was induced to have inflammatory bowel disease. Then, by threating the zebra fish with the compound represented by Chemical formula 1 of the present invention at different concentrations (0.3 μM and 1 μM), inhibitory activity on inflammatory bowel disease was examined.

As a zebra fish animal model of DSS-induced ulcerative colitis, zebra fish larvae 3 days after fertilization (3 dpf; days post fertilization) were used. 3 dpf zebra fish larvae were added to a 24-well plate, i.e., 20 larvae per well, and treated with 0.5% DSS to induce ulcerative colitis. After keeping them to 6 dpf zebra fish larva period, DSS was removed, and then they were treated for 5 hours with the compound represented by Chemical formula 1 of the present invention.

The zebra fish larvae which have been treated with the compound represented by Chemical formula 1 of the present invention were then added with TRIzol reagent (300 μl), and then homogenized by using a tissue homogenizer (G20, GINKO). Homogenized zebra fish larvae were maintained for 5 minutes at room temperature, and then treated with chloroform (60 μl). After removing proteins and cell debris, the mixture was centrifuged for 15 minutes at 12,000×g, 4° C. Transparent supernatant (100 μl) was transferred to a fresh microcentrifuge tube, added with the same amount of isopropanol, and vortexed for mixing. After keeping the mixture for 10 minutes at room temperature followed by centrifuge for 10 minutes at 12,000×g, 4° C., only the precipitated pellet was recovered with removal of the supernatant. After washing the precipitated pellet by treating it with 75% ethanol, the pellet was centrifuged again for 10 minutes at 12,000×g, 4° C. Supernatant was removed and the pellet was dried and dissolved in RNase free water (20 μl). Purity and concentration were measured by using a microplate reader (FLUOstar omega, BMG Labtech), and the extracted total RNA was kept at −80° C.

For cDNA synthesis, High-Capacity cDNA Reverse Transcriptase Kit (#4368814, Thermo Scientific) was used. The extracted total RNA (2 μg) in PCR tube (#4358293, Thermo Scientific) was added with 10× RT buffer solution (2 μl), 25× dNTP mix (0.8 μl), 10× RT random primer (2 μl), reverse transcriptase (1 μl), and RNase free water (4.2 μl), and cDNA was synthesized using a PCR device. As for the PCR conditions, the reaction was allowed to occur for 10 minutes at 25° C., 120 minutes at 37° C., and 5 minutes at 85° C. For the zebra fish animal model of DSS-induced ulcerative colitis, the activity of reverse transcriptase involved in cDNA synthesis is suppressed by residual DSS from RNA extraction process, and thus a washing step for completely removing DSS was additionally included in the RNA extraction process.

Specifically, to do so, Dynabeads™ mRNA DIRECT™ purification kit (#61012, Invitrogen) and DynaMag™-2 Magnet (#12321D, Invitrogen) were used. Magnetic beads (50 μl) included in the kit were aliquoted to a 1.5 ml microcentrifuge tube. By maintaining them for 30 seconds on a magnetic stand, magnetic beads in desired use amount were separated. To the separated magnetic beads, dissociation/binding buffer solution (150 μl) was added and mixed by vortexing. After adding the total extracted RNA, the mixture was maintained on a rotator for 5 minutes. After allowing the mixture to stand for 30 seconds on a magnetic stand, the solution except the magnetic beads adhered to the stand was removed. After separation from the magnetic stand, the mixture was washed with a washing buffer solution, and, by adding an elution buffer solution (20 μl), only mRNA was separated from the total RNA. cDNA synthesis from the purified mRNA was carried out by using TOPscriptm RT DryMix (dT18 plus, Enzynomics). cDNA was synthesized by using 2 μg of the extracted mRNA.

SYBR™ Green PCR master mix was used for qPCR reaction. qPCR conditions include pre-denaturation for 10 minutes at 95° C., and 40 cycles of denaturation step (30 seconds at 95° C.), annealing step (30 seconds at 55° C.), and extension step (30 seconds at 72° C.)

Primers (5′->3′) used for the experiment are as follows;

TNF-α_F:
(SEQ ID NO: 1)
ACAAGGCAATTTCACTTCCA,
TNF-α_R:
(SEQ ID NO: 2)
AGCTGATGTGCAAAGACACC;
IL-1β_F:
(SEQ ID NO: 3)
TTGTGGGAGACAGACGGTGC,
and
IL-1β_R:
(SEQ ID NO: 4)
GATTGGGGTTTGATGTGCTTCAT.

Transcription level of each gene was represented by relative increase/decrease using GAPDH as endogenous control.

The zebra fish animal model of DSS-induced inflammatory bowel disease was treated with the compound represented by Chemical formula 1 of the present invention, 5-ASA or steroid (prednisolone), which are currently used as a therapeutic agent for inflammatory bowel disease, and the inhibitory activity on proinflammatory cytokines was examined.

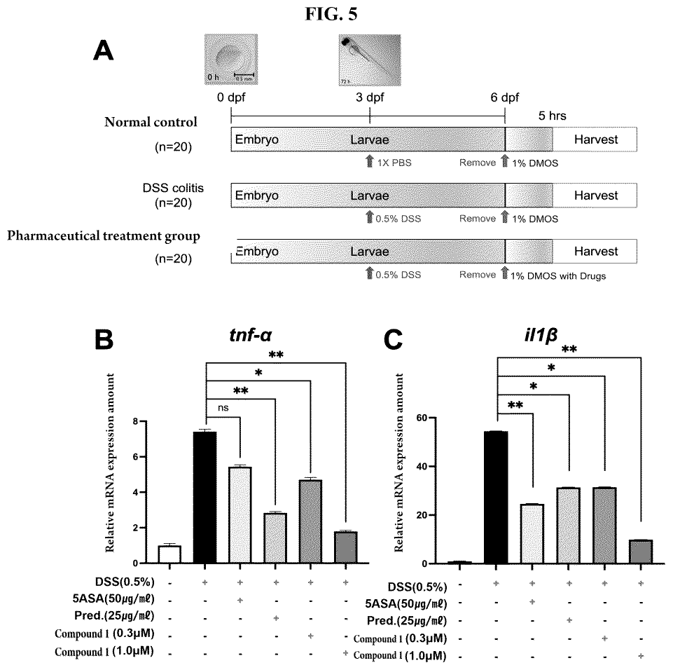

As a result, it was found that the compound represented by Chemical formula 1 of the present invention can reduce the transcription amount of proinflammatory cytokines at much lower concentration than 5-ASA and prednisolone (FIG. 5).

### Example 5. Evaluation of Therapeutic Effect Using Model of DSS-Induced Inflammatory Bowel Disease

As a mouse with induced inflammatory bowel disease, 6 week-old C57BL/6 was used, and the animal was induced to have the disease by administration of DSS (dextran sulfate sodium, MW 36000 to 50000, MP Biomedicals), Test groups were divided into the followings; normal control which has not been treated with any chemical (3 animals); group administered with DSS (6 animals); group administered with DSS+5-ASA (5-aminosalicylic acid, Sigma) (6 animals); and group administered with DSS+compound represented by Chemical formula 1 of the present invention (6 animals).

### (1) Evaluation Based on Disease Activity Index (DAI)

Inflammatory bowel disease is generally evaluated in terms of the disease activity index (DAI) based on clinical signs like body weight loss, diarrhea, and bloody feces. DAI is measured by giving a score to each of body weight loss rate, state of feces, and state of bloody feces (Table 1).

TABLE 1
Evaluation index for body weight loss,
feces state, and intestinal bleeding

| | Body weight | | |
|---|---|---|---|
| Score | decrease (%) | Stool consistency | Rectal bleeding |
| 0 | <1 | Normal | Normal |
| 1 | 1 to 5 | — | — |
| 2 | 5 to 10 | Watery feces not stuck to anus | Slight bleeding |
| 3 | 10 to 15 | — | — |
| 4 | >15 | Diarrhea stuck to anus | Visible bleeding |

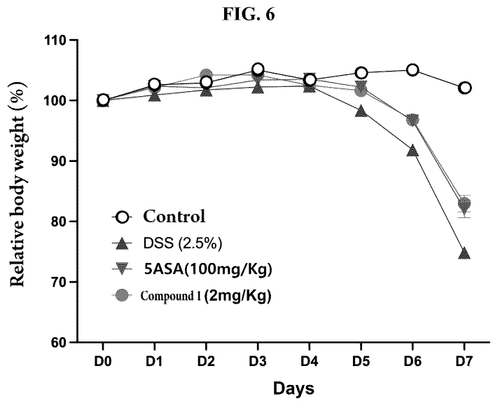

In this example, evaluation was made for 7 days for each test group. For all test groups except normal control, 100 ml of drinking water with 2.5% DSS were supplied for 7 days, while the water exchange was made every 2 days, to induce inflammatory bowel disease. The group administered with 5-ASA was orally administered, once a day for 7 days, with 100 mg/kg 5-ASA dispersed in 2.5% CMC (carboxymethylcellulose sodium, Sigma). The compound represented by Chemical formula 1 of the present invention (2 mg/kg) was administered via intraperitoneal injection, once a day for 7 days. From the first day of test, mouse of each test group was weighed to measure the rate of body weight loss. As a result, it was found that, till the last day of the test, body weight loss is not shown from normal control which has not been treated with any chemical. However, the weight loss was observed from Day 5 and thereafter in every test group induced to have inflammatory bowel disease by a treatment with DSS. It was found that, compared to the group administered with DSS, the group administered with DSS and 5-ASA and the group administered with DSS and the compound represented by Chemical formula 1 of the present invention exhibit lower rate of body weight loss (FIG. 6).

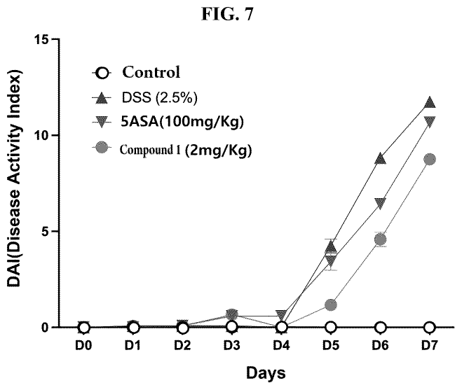

Evaluation of disease activity index (DAI) was also carried out, once a day from the first day of the test. From all test groups having inflammatory bowel disease induced by DSS treatment except normal control which has not been treated with any chemical, higher DAI was shown from Day 5 and thereafter. DSS treatment group showed the fastest increase in DAI followed by the group administered with DSS and 5-ASA and the group administered with DSS and the compound represented by Chemical formula 1 of the present invention in order (FIG. 7).

Sum of each item analyzed on Day 8 based on the evaluation index of Table 1 was described as disease activation index (DAI) in Table 2. It was found that the lowest DAI is obtained from the group administered with DSS and the compound represented by Chemical formula 1 of the present invention.

TABLE 2
DAI on Day 8 after administering for 7 days the compound represented
by Chemical formula 1 of the present invention

| Test group | DAI |
|---|---|
| Normal control | 0.00 |
| Group administered with DSS (2.5%) | 11.75 ± 0.15 |
| Group administered with DSS and Compound 1 | 8.75 ± 0.21 |
| (2 mg/kg, intraperitoneal administration) | |
| Group administered with DSS and 5-ASA (100 | 10.67 ± 0.19 |
| mg/kg, oral administration) | |

### (2) Measurement of Colon Length

With regard to the inflammatory response to inflammatory bowel disease, level of the response can be evaluated in terms of the decrease in colon length. Mouse in each test group was sacrificed on Day 8 and the colon was removed from the animal. Colon length was measured from the removed colon.

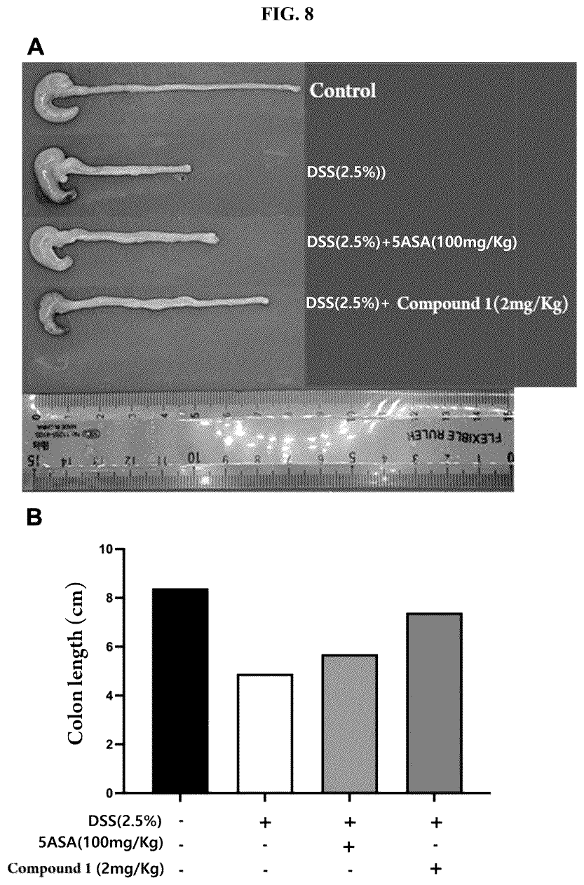

As the result is given in FIG. 8 and Table 3, the shortest colon length was shown from the DSS treatment group due to the inflammatory response caused by DSS, while longer colon length was shown from the group administered with DSS and 5-ASA and the group administered with DSS and the compound represented by Chemical formula 1 of the present invention in order.

TABLE 3
Determination of change in colon length after
administration of the compound represented by
Chemical formula 1 of the present invention

| Test group | Colon length (cm) |
|---|---|
| Normal control | 8.4 |
| Group administered with DSS (2.5%) | 4.9 |
| Group administered with DSS and Compound 1 (2 | 7.4 |
| mg/kg, intraperitoneal administration) | |
| Group administered with DSS and 5-ASA (100 | 5.7 |
| mg/kg, oral administration) | |

A sequence listing electronically submitted with the present application on Sep. 30, 2022 as an ASCII text file named 20220930_Q96822GR06_TU_SEQ.TXT, created on Sep. 20, 2022 and having a size of 1,027 bytes, is incorporated herein by reference in its entirety.

1-8. (canceled)

9: A method for treating inflammatory bowel disease, the method comprising:
preparing a composition comprising a compound represented by Chemical Formula 1 or a pharmaceutically or sitologically acceptable salt thereof as an effective component: 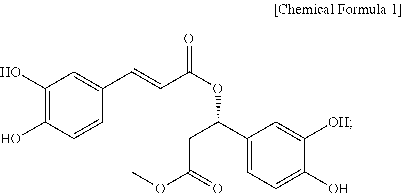 [SMILES: COC(=O)C[C@H](OC(=O)/C=C/c1ccc(O)c(O)c1)c1ccc(O)c(O)c1]
and
administering the composition to a subject in need thereof.

10: The method of claim 9, wherein the inflammatory bowel disease is selected from the group consisting of ulcerative colitis, Crohn's disease, ischemic colitis, intestinal Behcet's disease, bleeding rectal ulcer, and pouchitis.

11: The method of claim 9, wherein the inflammatory bowel disease is ulcerative colitis.

12: The method of claim 9, wherein the inflammatory bowel disease is Crohn's disease.

13: The method of claim 9, wherein the inflammatory bowel disease is ischemic colitis.

14: The method of claim 9, wherein the inflammatory bowel disease is intestinal Behcet's disease.

15: The method of claim 9, wherein the inflammatory bowel disease is bleeding rectal ulcer.

16: The method of claim 9, wherein the inflammatory bowel disease is pouchitis.

17: The method of claim 9, wherein the compound represented by Chemical Formula 1 is isolated from Isodon excisus.

18: The method of claim 9, wherein the pharmaceutically acceptable salt is an inorganic acid selected from hydrochloric acid, phosphoric acid, sulfuric acid, nitric acid, and tartaric acid.

19: The method of claim 9, wherein the pharmaceutically acceptable salt is an organic acid selected from methane sulfonic acid, p-toluene sulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid.

20: The method of claim 9, wherein the pharmaceutically acceptable salt is a non-toxic salt selected from sulfate, pyrosulfate, bisulfate, sulfite, bisulfite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, and mandelate.

21: The method of claim 9, wherein the composition further comprises a pharmaceutically acceptable carrier, vehicle, or diluent in addition to the effective component.

22: The method of claim 9, wherein the composition is a functional health food composition prepared in a formulation selected from powder, granule, pill, tablet, capsule, candy, syrup, and beverage.

23: The method of claim 9, wherein the composition is an animal feed composition.
